(19) United States
(12) Patent Application Publication
(10) Pub. No.: US 2019/0110696 A1
(43) Pub. Date: Apr. 18, 2019

(54) VASCULAR MONITORING SYSTEM

(71) Applicant: DESIGNPLEX BIOMEDICAL, LLC, Fort Lee, NJ (US)

(72) Inventors: Robert BENKOWSKI, Fort Lee, NJ (US); Gino MORELLO, Fort Lee, NJ (US); Sivaprasad SUKAVANESHVAR, Fort Lee, NJ (US)

(21) Appl. No.: 16/090,003

(22) PCT Filed: Mar. 31, 2017
(86) PCT No.: PCT/US2017/025522

§ 371 Date: Sep. 28, 2018

Related U.S. Application Data
(60) Provisional application No. 62/315,997, filed on Mar. 31, 2016.

(51) Int. Cl. A61B 5/02; A61B 5/026; A61B 5/00

(52) U.S. Cl. CPC A61B 5/02007; A61B 5/0261; A61B 5/0031; A61B 5/0086; A61B 5/6852; A61B 2560/0204; A61B 5/686

(57) ABSTRACT

A vascular sensor system includes a vascular sensor configured to monitor a blood vessel parameter and to output a signal representative of the measured parameter. A computing system is configured to receive the signal representative of the measured parameter. The computing system is programmed to process the received signal and evaluate a medical condition based on the processed signal.

This application is being filed on 31 Mar. 2017, as a PCT International Patent application and claims the benefit of priority to U.S. Provisional patent application Ser. No. 62/315,997, filed Mar. 31, 2016, the entire disclosure of which is incorporated by reference in its entirety.

### BACKGROUND

This disclosure relates generally to a vascular monitoring system.

According to the Centers for Disease Control (CDC), the precise number of people affected by deep vein thrombosis (DVT) and pulmonary embolism (PE) is unknown, although as many as 900,000 people could be affected (1 to 2 per 1,000) each year in the United States. Estimates suggest that 60,000-100,000 Americans die of DVT/PE (also called venous thromboembolism or VTE) per year, and 10 to 30% of people will die within one month of diagnosis. Sudden death is the first symptom in about one-quarter (25%) of people who have a PE. Among people who have had a DVT, one-half will have long-term complications (post-thrombotic syndrome) such as swelling, pain, discoloration, and scaling in the affected limb. About one-third of people with DVT/PE will have a recurrence within 10 years. Approximately 5 to 8% of the U.S. population has one of several genetic risk factors, also known as inherited thrombophilias, in which a genetic defect can be identified that increases the risk for thrombosis.

VTE is a disorder that can occur in all races and ethnicities, all age groups, and both genders. With many of the known risk factors-advanced age, immobility, surgery, obesity-increasing in society, VTE is an important and growing public health problem. Recently, a marked increase has occurred in federal and national efforts to raise awareness and acknowledge the need for VTE prevention. Yet, many basic public health functions-surveillance, research, and awareness-are still needed. Learning and understanding more about the burden and causes of VTE, and raising awareness among the public and healthcare providers through a comprehensive public health approach, has enormous potential to prevent and reduce death and morbidity from deep vein thrombosis and pulmonary embolism throughout the U.S. Pulmonary embolism is a blockage in one of the pulmonary arteries in one's lungs. In most cases, pulmonary embolism is caused by blood clots that travel to the lungs from the legs or, rarely, other parts of the body (e.g. deep vein thrombosis). Because pulmonary embolism almost always occurs in conjunction with deep vein thrombosis, most doctors refer to the two conditions together as venous thromboembolism. Although anyone can develop DVT and PE, factors such as immobility, cancer and surgery increase your risk.

While conditions such as DVT/PE can be life-threatening, prompt treatment can greatly reduce the risk of death. Prevention is the key to reducing death and disability resulting from VTE. This includes thromboprophylaxis in patients at risk (primary prevention), such as those undergoing surgery or those hospitalized with medical illnesses, and prevention of recurrent thromboembolic events in patients with established DVT or PE (secondary prevention). Effective primary prevention is available for most high-risk patient groups. However, a global audit of utilization of primary thromboprophylaxis showed widespread underuse in eligible patients. There is evidence that a concerted effort by a health system to include VTE risk assessment at the time of hospital admission and the provision of appropriate primary thromboprophylaxis is effective in reducing the frequencies of VTE-related death and readmission with non-fatal VTE. The increased implementation of proven, evidence-based primary prevention of VTE should be a global health priority. The safety and simplicity of extended anticoagulant therapy have improved significantly in recent years, and this approach to secondary prevention has the potential to markedly reduce the burden caused by recurrent VTE events if appropriately implemented on a global scale. Strengthening the global effort to prevent VTE is consistent with the World Health Assembly's goal of significantly reducing the global burden caused by non-communicable diseases by 2025. In conclusion, this literature review found substantial evidence of a major global disease burden caused by VTE. Although this burden has been less extensively evaluated than the burden caused by arterial thrombosis, which includes ischemic heart disease and ischemic stroke, the available evidence indicates a major burden of disease across low-income, middle-income and high-income countries. Because many of these events are potentially preventable, more detailed data on the burden caused by VTE should be obtained to inform public health policy and resource.

A stroke is any sudden event affecting the brain's blood supply. The most common type, almost 80% of all strokes, is ischemic stroke, where the blood supply to the brain is cut off or severely reduced due to a blocked artery. A condition known as stenosis contributes to an individual's risk for this type of stroke.

Stenosis, in general, refers to any condition in which a blood vessel—such as an artery—or other tubular organ becomes abnormally narrow. In the context of stroke, stenosis is usually caused by atherosclerosis, a condition where a blood vessel supplying blood to the brain is narrowed due to fatty deposits, known as plaques, on the vessel's inside wall. Risk factors for this type of stenosis include high blood pressure and high cholesterol.

Atherosclerosis can activate cells involved in blood clotting. As clots form, they can obstruct narrowed blood vessels in the neck (the carotid artery) or the small blood vessels of the brain (intracranial arteries). Additionally, a clot or piece of the plaque can break free and flow to the brain and block an artery.

Atherosclerosis, sometimes called hardening of the arteries, can slowly narrow and harden the arteries throughout the body. When atherosclerosis affects the arteries of the heart, it's called coronary artery disease.

Coronary artery disease is the No. 1 killer of Americans. Most of these deaths are from heart attacks caused by sudden blood clots in the heart's arteries.

The following statistics were supplied by WebMD:

More than 15,800,000 Americans have known coronary artery disease.

About 8 million of them have had heart attacks.

Around 500,000 people will die of coronary artery disease this year. More than a million will have a heart attack.

One-third of all deaths in Americans older than 35 are due to coronary artery disease.

After age 40, about 50% of men and one-third of women can expect to eventually have coronary artery disease.

Arterial embolism is a sudden interruption of blood flow to an organ or body part due to an embolus adhering to the wall of an artery blocking the flow of blood, the major type of embolus being a blood clot (thromboembolism). Sometimes, pulmonary embolism is classified as arterial embolism as well, in the sense that the clot follows the pulmonary artery carrying deoxygenated blood away from the heart. However, pulmonary embolism is generally classified as a form of venous embolism, because the embolus forms in veins. Arterial embolism is the major cause of infarction (which may also be caused by e.g. arterial compression, rupture or pathological vasoconstriction).

Surgical and intensive care patients are at a heightened risk for arterial embolization due to pre-existing conditions such as age, hypercoagulability, cardiac abnormalities and atherosclerotic disease. Most arterial emboli are clots that originate in the heart and travel to distant vascular beds where they cause arterial occlusion, ischemia, and potentially infarction. Other emboli form on the surface of eroded arterial plaque or within its lipid core. Thromboemboli are large clots that dislodge from the surface of athesclerotic lesions and occlude distal arteries causing immediate ischemia. Atheroemboli, which originate from fracturing the lipid core tend to cause a process of organ dysfunction and systemic inflammation, termed cholesterol embolization syndrome. The presentation of arterial emboli depends on the arterial bed that is affected. The most common manifestations are strokes and acute lower limb ischemia. Less frequently, emboli target the upper extremities, mesenteric or renal arteries.

According to NIH, recent mortality rates due to arterial emboli range from 4 to 15%. As expected, patients with arterial emboli have multiple risk factors for perioperative morbidity and mortality. One typical patient population studied had an average age of 69 years. Within this group 55% of patients had peripheral arterial disease, 47% had coronary artery disease, 28% had cerebrovascular disease, 46% had diabetes mellitus, 56% had HTN, 40% had hyperlipidemia and 69% used tobacco. Other predictors of morbidity and mortality include coexisting bowel ischemia, poor preoperative functional status, cardiac insufficiency and renal disease. Causes of death included myocardial infarction (MI) and other cardiac complications, pneumonia, renal failure and sepsis with multi-organ-system failure. Morbidities outside of limb loss included heart failure, MI, stroke, respiratory failure, renal insufficiency, pulmonary emboli, bowel ischemia and infections.

An aortic aneurysm is a balloon-like bulge in the aorta, the large artery that carries blood from the heart through the chest and torso.

According to CDC, Aortic aneurysms were the primary cause of 10,597 deaths and a contributing cause in more than 17,215 deaths in the United States in 2009. About two-thirds of people who have an aortic dissection are male. The U.S. Preventive Services Task Force recommends that men aged 65-75 years who have ever smoked should get an ultrasound screening for abdominal aortic aneurysms, even if they have no symptoms.

### SUMMARY

In accordance with certain aspects of the present disclosure, a vascular monitoring system includes a vascular sensor configured to monitor a parameter of a blood vessel. For example, the sensor may monitor blood flow through a blood vessel and provide sensor output via a communication link to a computing system, such as an external monitoring system. The sensor output may be configured for real-time output or for periodic output for energy conservation. In some examples, one or more sensors are implanted into the body of a patient at an implant site or multiple implant sites of concern. The computing system is programmed to process the received sensor output and evaluate a medical condition based on the processed signal.

### BRIEF DESCRIPTION OF THE DRAWINGS

FIG. 1 is a block diagram conceptually illustrating aspects of a vascular monitoring system in accordance with the present disclosure.

FIG. 2 is a block diagram illustrating an example of portions of the computing system shown in FIG. 1.

FIG. 3 illustrates portions of an implantable vascular sensor in accordance with the present disclosure.

FIG. 4 illustrates arterial tissue with no light source present

FIG. 5 depicts the same arterial tissue in FIG. 4 illuminated from behind with a visible light source.

FIG. 6 illustrates venous tissue with no light source present.

FIG. 7 depicts the same venous tissue in FIG. 6 illuminated from behind with a visible light source.

FIG. 8 depicts a carotid placement of the vascular sensor shown in FIG. 1.

FIG. 9 illustrates an example baseline sensor output signal with no clot formations present in the blood stream.

FIG. 10 and FIG. 11 illustrate an example of the sensor's output signal with a single thromboembolism present in the blood stream.

FIG. 12 and FIG. 13 illustrate an example of the sensor's output signal with a multiple thromboemboli present in the blood stream.

FIG. 14 illustrates exemplary placement of a clot detector sensor for detection of Deep Vein Thrombosis.

FIG. 15 and FIG. 16 illustrate an example of the sensor's output signal, acquired across the wall of a vein, with Normal Flowing Blood versus Static/Clotted Blood (DVT).

FIGS. 17A and 17B illustrate an example of the sensor's output signal for Intimal Thickening (Stenosis) & Plaque.

FIG. 18 illustrates an exemplary placement of a clot detector sensor for A-V graft stenosis.

FIG. 19 illustrates diagrammatically an example of an A-V graft stenosis.

FIG. 20 illustrates an example of a conceptual topology of a typical remote monitoring system.

FIG. 21 illustrates an example of an operational unit on the network.

FIG. 22 illustrates an exemplary data center topology.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as top, bottom, front, back, etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense.

This disclosure relates generally to a vascular monitoring system. Some examples of the disclosed vascular monitoring system provide an early and immediate indication of vascular pathology. By detecting such pathology as they happen and providing this information to the patient and a remote monitoring center, for example, before the patient may become symptomatic should greatly improve patient survivability.

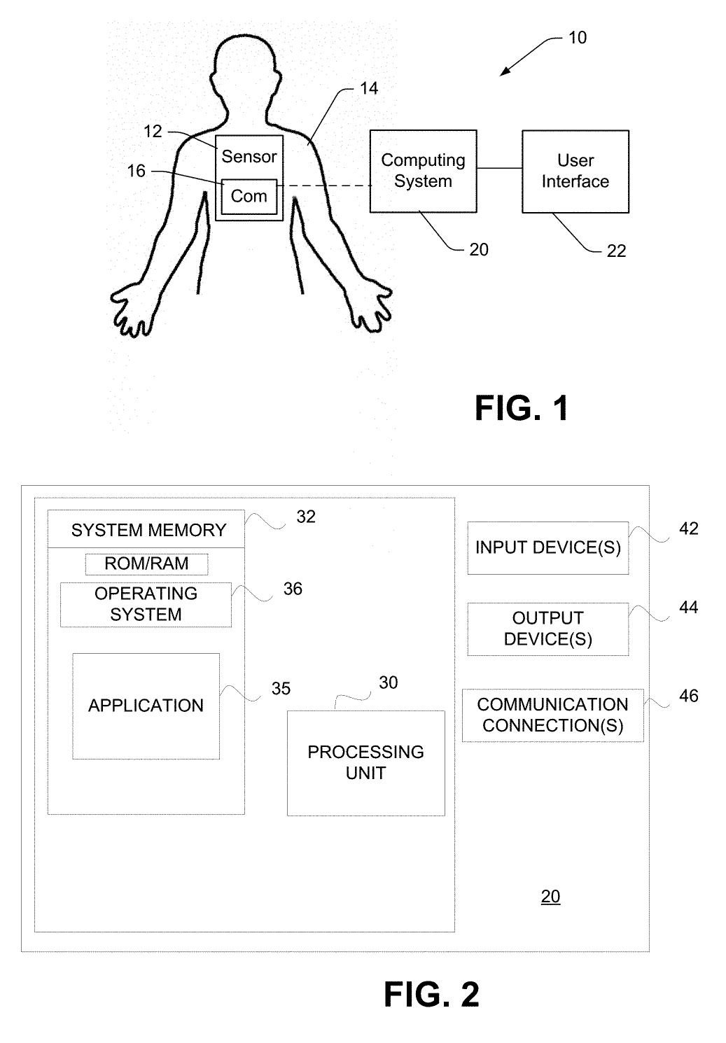

FIG. 1 conceptually illustrates an example of an implantable sensor system in accordance with disclosed embodiments. The system 10 includes an implantable vascular sensor 12, shown implanted in a patient 14. The sensor 12 is configured to monitor a blood vessel parameter and to output a signal representative of the measured parameter, which is received by a computing system 20 that is programmed to process the received signal and evaluate a medical condition based on the processed signal is provided. The computing system 20 generates a user interface 22 to provide information regarding the medical condition for the patient or other user.

FIG. 2 is a simplified block diagram illustrating aspects of an example of the computing system 20. In a basic configuration, the computing device 20 includes at least one processor or processing unit 30 and a system memory 32. Depending on the configuration and type of computing device, system memory 34 may comprise, but is not limited to, volatile (e.g. random access memory (RAM)), non-volatile (e.g. read-only memory (ROM)), flash memory, or any combination. The system memory 32 may include an operating system 36 and software code for implementing various applications 35.

The computing system may also include additional data storage devices (not shown) that may be removable and/or non-removable such as, for example, magnetic disks, optical disks, solid state storage devices (“SSD”), flash memory or tape. The computing system 20 may also have input device(s) 42 such as a keyboard, a mouse, a pen, a sound input device (e.g., a microphone), a touch input device, etc. Output device(s) 44 such as a touchscreen display, speakers, a printer, etc. may also be included. The input and/or output devices 42, 44 may be configured to provide the user interface 22 shown in FIG. 1. Communication connection(s) 46 may also be included and utilized to connect to the networks such as the internet, as well as to remote computing systems such as server computer or other networked devices.

Aspects if the various embodiments disclosed, for example, may be implemented as a computer process (method), a computing system, or as an article of manufacture, such as a computer program product or computer readable media. The computer program product may be a computer storage media readable by a computer system and encoding a computer program of instructions for executing a computer process.

The term computer readable media as used herein may include computer storage media. Computer storage media may include volatile and nonvolatile, removable and non-removable media implemented in any method or technology for storage of information (such as computer readable instructions, data structures, program modules, or other data) in hardware. The system memory 2104 is an example of computer storage media (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by the computing system 20. Any such computer storage media may also be part of the computing device 20. Computer storage media does not include a carrier wave or other propagated or modulated data signal.

The term computer readable media as used herein may also include communication media. Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Emboli, Flowing blood, Static/Clotted Blood, normal blood vessel wall, thickened blood vessel wall, etc. will elicit distinct optical signatures which may be detected by the implantable sensor 12. Sensor configuration and signal processing/analysis permits differentiation of these signatures. In this manner, the disclosed system shifts the clinical paradigm from reactive to proactive medicine, helping to minimize unpredictability and possibly prevents potentially catastrophic events.

Thus, embodiments of the disclosed system 10 enable examination of macroscopic events that have direct/established clinical consequences. For example, the implantable sensor 12 may be configured for monitoring parameters relating to conditions such as vessel wall disease, thrombosis and emboli. The implantable sensor 12 further includes a communications device 16 to transmit sensed data from the sensor 12 to an external patient monitoring system implemented by the computing system 20, periodically or in real-time. Some examples further include additional communications ability for further transmitting said data over a network such as the internet to a remote monitoring system or station for recording, processing, evaluation, and disposition.

Various examples of the system 10 provide capabilities for identifying and evaluating the monitored parameters to identify conditions such as:
Embolic Stroke due to Cardiac Arrhythmia, Valvular issues, Carotid plaques, Peri-procedural issues associated with Transcatheter Aortic Valve Implantation (TAVI) for Degenerated Bioprosthetic Heart Valves, Radio Frequency (RF) ablation, Thrombectomies, mechanical clot retrieval, stroke complication after coronary artery bypass grafting involving Coronary Artery Bypass Graft (CABG) with extracorporeal circulation (ECC), Left Ventricular Assist Devices (LVAD), and Total Artificial Hearts (TAH).
Deep Vein Thrombosis (DVT) and Pulmonary Embolism (PE), Lower & Upper extremity (e.g. Femoral, Popliteal, Iliac & Subclavian/brachiocephalic veins
Intimal Hyperplasia (Stenosis), A-V graft for hemodialysis Vascular Access, Coronary artery bypass graft
Atherosclerotic Plaque, Coronary Artery Disease, Peripheral Artery Disease
Cancer and Tumor Growth, characterized by in an increase in vascularization
Aneurysm detection, characterized by detecting the thinning or weakening of the vessel wall. One application is Abdominal Aortic Aneurysms (AAA).

In some examples, the computing system 20 configured for processing of sensor data, among other things, is a patient worn device. In another embodiment, the sensor data is transmitted over a network such as the Internet for processing and evaluation by a clinician or clinical bureau remotely from the sensor 12 and patient 14. The sensor 12 may be configured to run continually or periodically.

The sensor 12 may comprise a single sensor or multi-sensor array configured as a small implantable network. In some implementations, the sensor 12 is internally powered by a primary or secondary battery, and in other examples it is passively powered by an external instrument. Still further embodiments use vessel pulsatility to generate power to run the sensor 12 through motion harvesting.

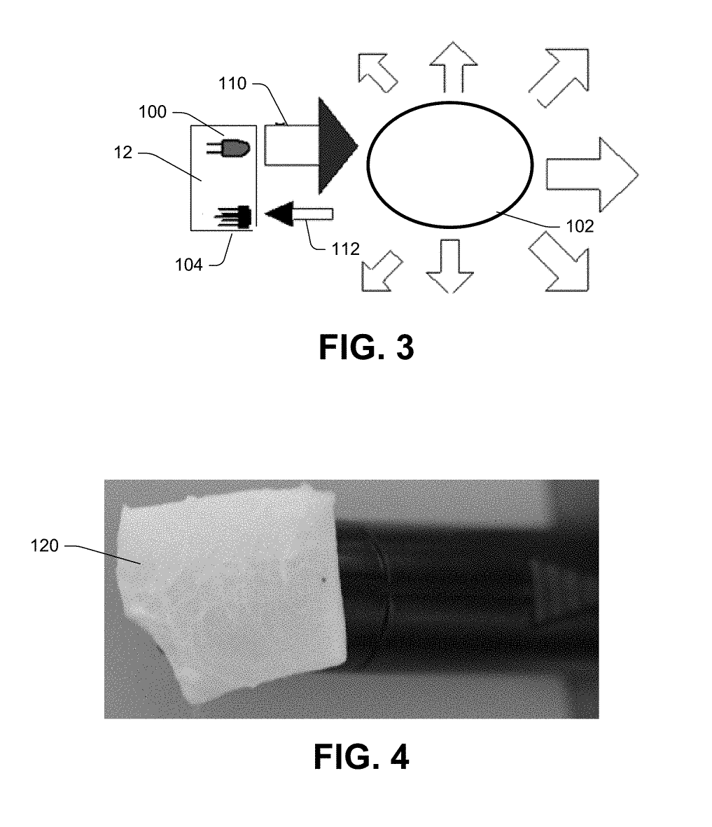

The sensor 12 may be implanted into the patient 14 to detect certain pathologies in the blood vessel and in the bloodstream as noted previously. Although several mechanisms for detection may be used, the detection sensor in one embodiment is based on a Wave Source-Signal Detector utilizing Near Infrared (NIR) light as depicted in FIG. 3. Light 110 is emitted from a light source 100 (e.g. Light Emitting Diode (LED), LASER, or similar emitter source) such it “illuminates” the vessel of concern 102. As blood cells and potential emboli flow past the sensor 12, the light 110 generated by the light source 100 is reflected and/or scattered by the monitored vessel 102, and the reflected or scattered light 112 is detected by a photodetector 104 (e.g. photo transistor, photo diode, receptor, etc.). The resulting signal generated by the reflected/scattered light is then acquired, processed using a variety of algorithms in the time, frequency, and/or phasor domains, and an indication of whether or not an alteration associated with a pathology (e.g. clot, vessel wall disease) is detected. The result may then be transmitted using RF or other means (e.g. ultrasound, light, or via inductive coupling) to an external patient monitor with remote connectivity to alert the patient or monitoring bureau such that the patient may then rush to a hospital or clinic for immediate care.

In some implementations, PWM control of the light emitter 100 is employed to lower overall sensor power and to maximize implant life. Automatic light emitter bias control may be used to ensure the receptor 104 always receives an adequate reflected signal amplitude to ensure good signal-to-noise ratio (SNR). Automatic light emitter bias control may be used to ensure that the minimal amount of light output is needed for signal reception in order to maximize battery life and not unnecessarily overdrive the light emitter.

In various embodiments, the received signals can be instantaneous signals for detecting conditions such as a blood clot going to a major organ (brain, lungs, kidney, heart, etc.), or they could be longer term (days or weeks) of signal changing due to a build-up of clot, indicating conditions such as deep vein thrombosis, vascular stenosis, atherosclerosis, aneurysm, etc.

The light source 100 could output visible spectrum light or non-visible spectrum light. The sensor 12 could be implanted device in any number of locations, such as around an artery or vein, which could include the pulmonary artery, aortic arch, femoral vein, carotid artery, etc. In some embodiments, the sensor 12 could be an external device configured to monitor the carotid artery, for example.

The sensor 12 may provide raw signal out to the computing system 20, or the sensor 12 could include on-board signal processing. The user interface 22 is configured to alert the patient or clinicians regarding monitored conditions. The user interface 22 may be a native component of the computing system 20, or it could be implemented on an external device such as a smart phone app.

As noted above, in some examples sensor data is first transmitted through the body to a patient worn transceiver which may be programmed to process said data with expert algorithms and provide indication to the patient through the user interface 22 as to the presence of a condition such as a clot such that he/she can immediately seek clinical care. In other examples, sensor data is first transmitted through the body to a patient worn transceiver and subsequently may be transmitted further through a wired or wireless intranet or internet connection to a remote station for processing, recording, evaluation, and disposition by a set of expert algorithms, trained clinical expert, or a combination thereof.

Time domain analysis including amplitude threshold, peak-to-average (P/AR), and peak-to-rms (Crest Factor) detection algorithms may be employed for detecting occurrence and number of blood clots. Frequency domain analysis and Phasor domain analysis are also employed in some embodiments.

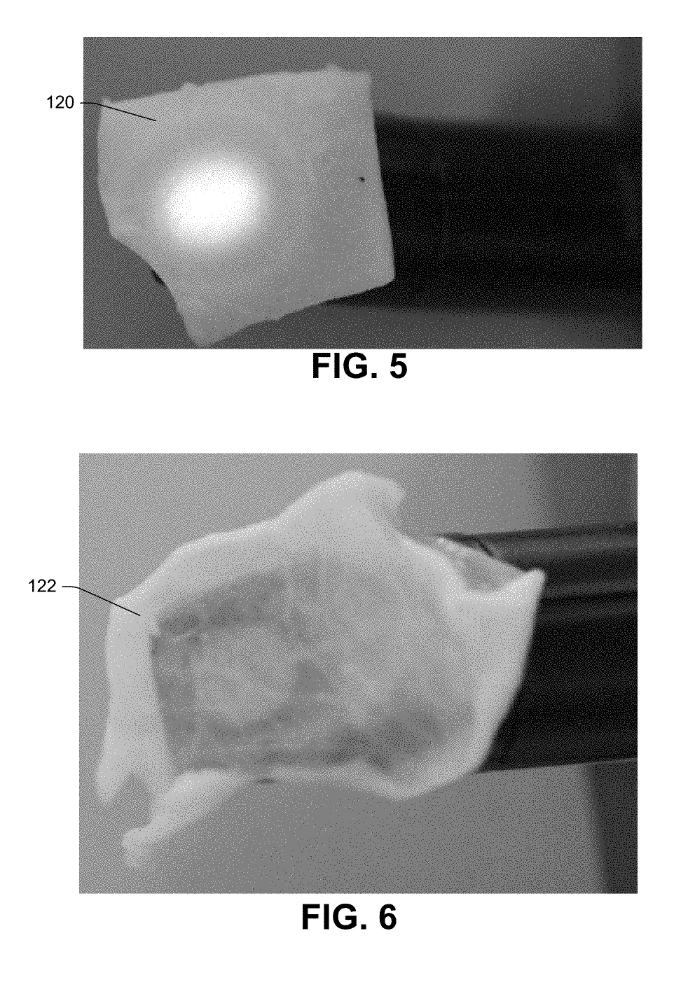

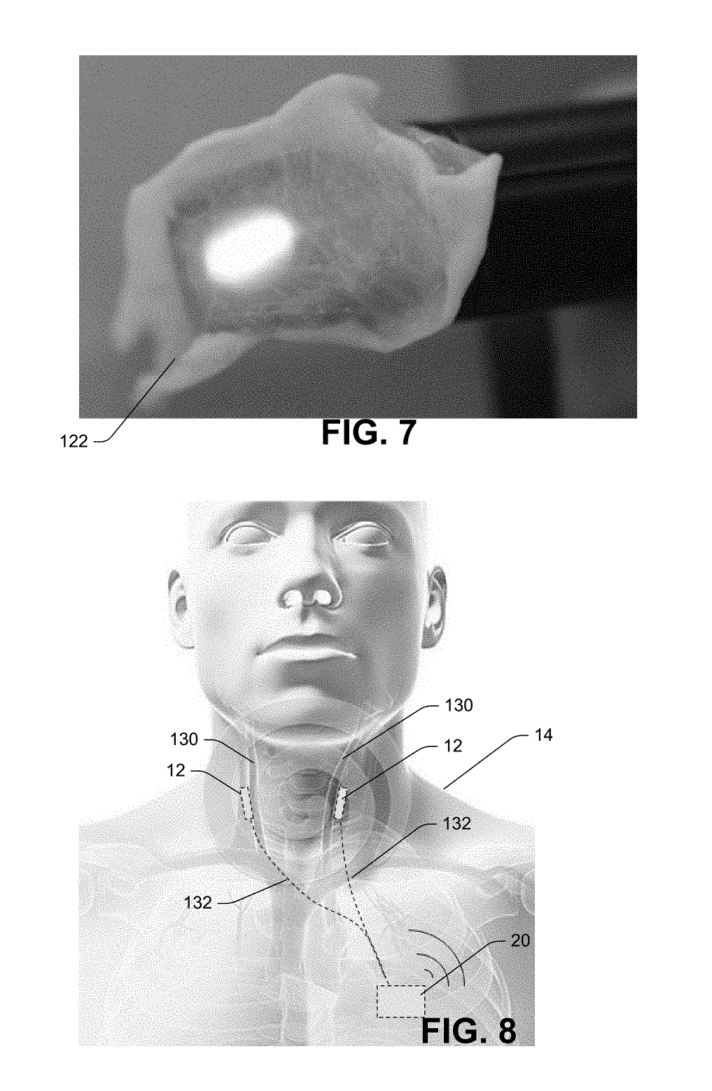

Although several modalities may be used to detect the presence of emboli, one embodiment, such as the sensor 12 illustrated in FIG. 3, is based on light since both arteries and veins are translucent and lend themselves well for the transmittance of near infrared light. FIG. 4 illustrates arterial tissue 120 with no light source present while FIG. 5 depicts the same arterial tissue 120 in FIG. 4 illuminated from behind with a visible light source near 640 nm (red light). FIG. 6 illustrates venous tissue 122 with no light source present while FIG. 7 depicts the same venous tissue illuminated from behind with a visible light source near 640 nm (red light). Thus, in FIG. 3, the light source 100 and the photodetector 104 are situated on the same side of the monitored vessel 102. Light output from the light source 100 is scattered by the vessel 102, and back-scattered light is received by the photodetector 104. The signal output by the photodetector 104 representative of the received light is received and processed by the computing system 20. In other embodiments, the light source 100 and photodetector 104 are positioned on opposite sides of the monitored vessel 102

Thromboembolic event types may be characterized by a unique sensor output signal wave shape. It is this signal wave shape that allows the system to not only detect the occurrence of a clot, but the type of clot and the number of clots. The information may then be acted upon in a specific manner by an attending clinician to ensure an optimal patient outcome. Representative signal characteristics of actual thromboemboli signals acquired across the wall of a carotid artery as observed by an exemplary optical sensor have been included herein for reference. FIG. 8 depicts an example of carotid placement for monitoring of cardiac emboli leading to ischemic cerebrovascular stroke, in which sensors 12 are implanted and positioned adjacent the carotid arteries 130. In the example illustrated in FIG. 8, tunneled leads 132 connect the sensors 12 to the computing system 20, which is an implantable, battery-powered signal processing unit in some implementations. In other examples, the sensors 12 are configured to wirelessly communicate with the computing system 20. The implanted computing system 20 includes the communication interface 16 such that it can communicate with a receiving unit external to the body. The receiving unit may be configured to provide the user interface 22.

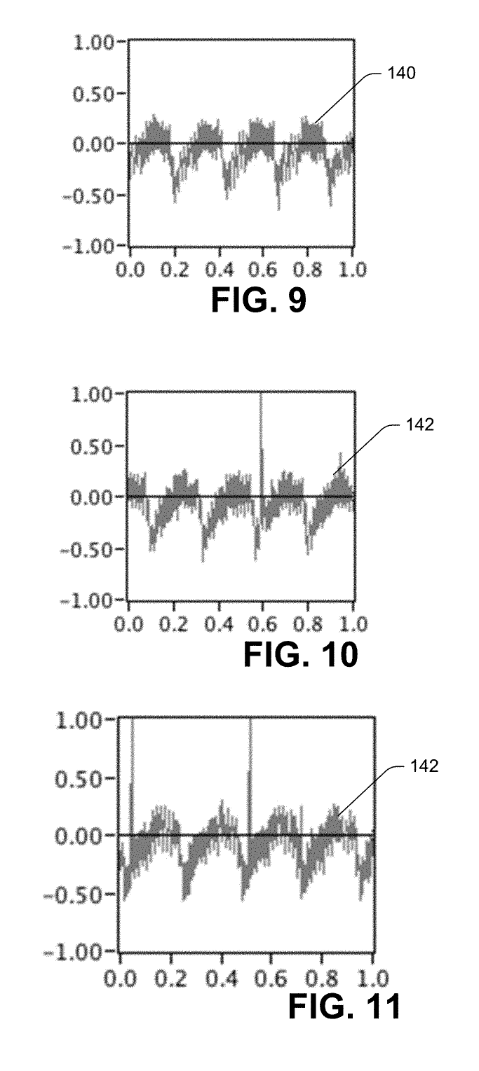

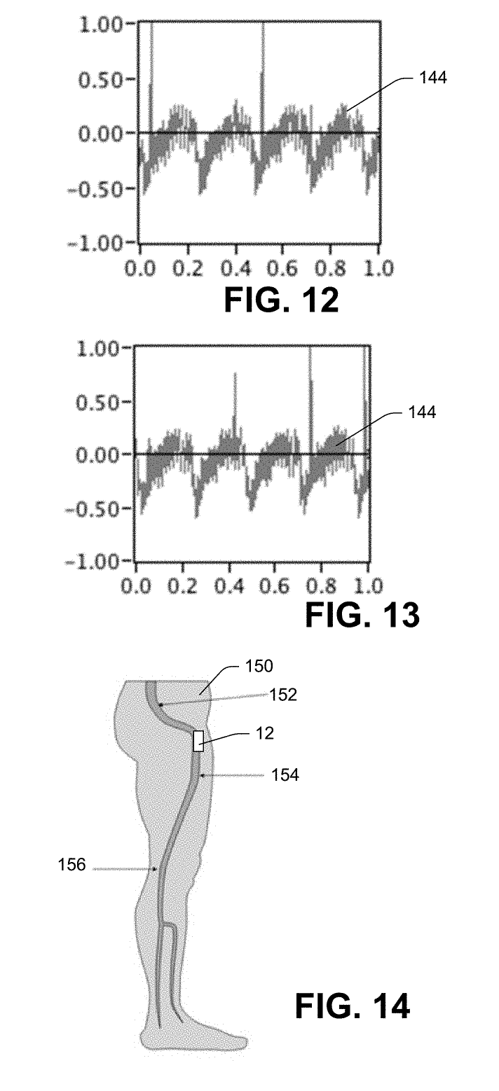

FIG. 9 shows an example of a baseline sensor output signal 140 with no emboli present in the blood stream. FIGS. 10 and 11 illustrate examples of the sensor's output signal 142 when a single thromboembolus is present in the blood stream. FIGS. 12 and 13 detail the sensor's output signal 144 with a multiple thromboemboli present in the blood stream. FIG. 14 illustrates exemplary placement of a clot detector sensor for detection of Deep Vein Thrombosis (DVT). The illustrated leg 150 of the patient 14 includes the iliac vein 152, the femoral vein 154 and the popliteal veins 156. For example, the sensor 12 could be implanted adjacent the femoral vein 152 to detect or predict DVT.

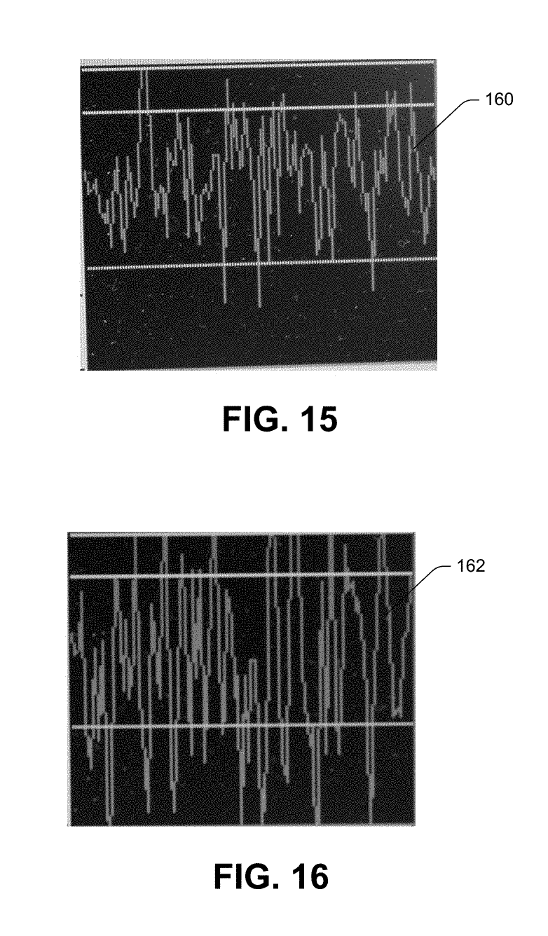

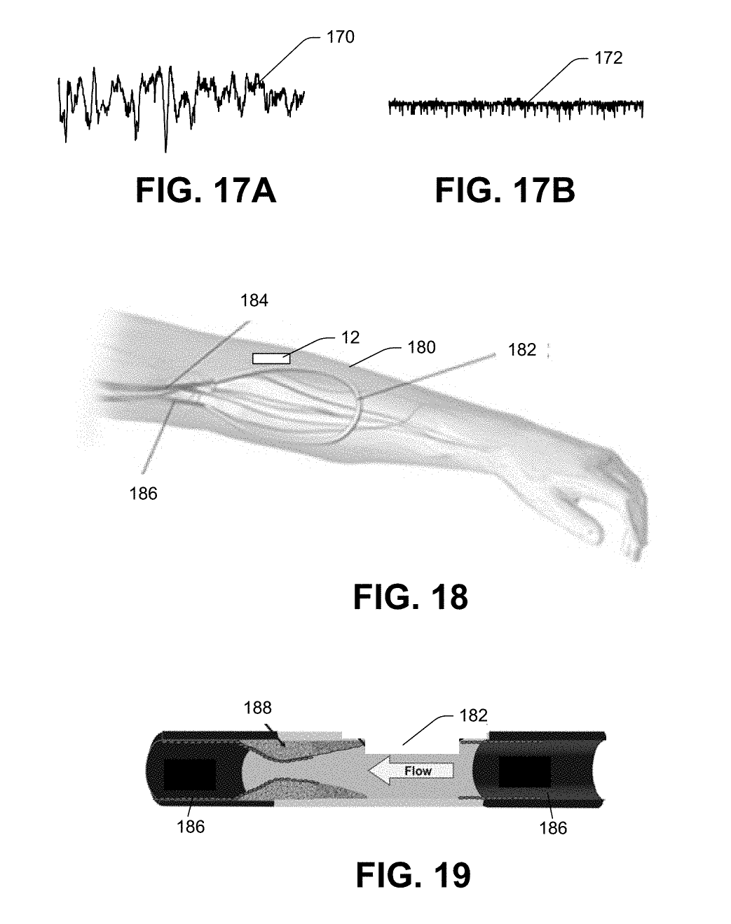

FIG. 15 and FIG. 16 show examples of the sensor's output signal, acquired across the wall of a vein, with normal flowing blood versus static/clotted Blood (DVT). FIG. 15 shows the output signal 160 with normal flowing blood, while FIG. 16 shows the output signal 162 with static/clotted blood, which may indicate a DVT. FIGS. 17A and 17B shows an example of the sensor's output signal for Intimal Thickening (Stenosis) & Plaque. The waveform 170 of FIG. 17A shows the sensor's output signal from blood flow across the wall of a normal blood vessel while the waveform 172 of FIG. 17B shows the sensor's output signal attenuated blood flow signal across the wall of thickened blood vessel.

FIG. 18 illustrates exemplary placement of a clot detector sensor for A-V graft stenosis, in which the sensor 12 is implanted in the patient's arm 180 adjacent an A-V graft 182 that connects an artery 184 and a vein 186. FIG. 19 illustrates diagrammatically an example of an A-V graft stenosis 188.

The sensor 12 may be utilized in an Extravascular manner (increased FBR risk) for Short-term (peri-procedural emboli) and Long-term (other emboli, DVT, intimal thickening & plaque) indications. The sensor may be utilized in an Intravascular manner (may be associated with increased thrombosis risk) Short-term (peri-procedural emboli) and Long-term (other emboli, DVT, intimal thickening & plaque): Smart Stents indications. The sensor may be configured for Continuous sensing for Emboli detection or, the sensor may be configured for Intermittent sensing DVT, Intimal thickening, and plaque indications.

Once a sensor 12 is implanted it continuously or intermittently communicates with an external patient device that, among other things, provides the user interface 22. The external device may be a dedicated receiver or transceiver or another device such as the patient's smartphone. In one embodiment a low power bidirectional communication link is established between the implant and external receiver. The frequency(ies) and output power used may be in the ISM, MICS, or other band suitable for short range, low power, and bidirectional communication.

In the exemplary embodiment the MICS band is implemented. MICS is an acronym for Medical Implant Communication Service. The band extends from 402-405 MHz and was designed and approved expressly for short-range, wireless link to connect low-power implanted medical devices with monitoring and control equipment Implanted Medical Devices (IMD) such as cardiac pacemakers, implantable cardioverter/defibrillator (ICD), neurostimulators, etc. The band plan of 402-405 MHz was selected as it provides reasonable signal propagation characteristics in the human body and has general world-wide acceptance and is approved in the United States, Europe, Canada, Australia and Japan.

Traditional implants use inductive links with limited range and required the telemetry receiver to be in contact with patient. Because they operated at low frequency they were only capable of achieving data rates similar to a dial-up computer modem. The former systems were not user friendly for home monitoring as they required a wand to be positioned above the IMD by the patient. Thus, there was a need for higher data rates to upload patient events captured in the IMD's memory to the base station for analysis. Higher data rates would also shorten doctor/patient consultancy times.

MICS transceivers typically can achieve data rates up to 800 kbits/second, require less than 250 nA when in sleep mode and less than 1 mA active currents, with a range of up to 2 meters. In one embodiment, a Zarlink ZL70101 MICS transceiver is used to transmit data from the clot sensors to an external device.

In some examples, the computing device 20, which may be a patient worn transceiver, is programmed to implement modules for data compression, encoding algorithms and proprietary packet structures to allow the device to transmit information, in normal conditions, at approximately 1 packet per second. The information to be transmitted shall include includes operational variables.

The encoding/compressing process was designed to achieve three goals: (1) To optimize transmission performance by reducing packet size. (2) To prevent unauthorized access of information contained in the packet during transit time, by encrypting contents beyond recognition by outsiders. (3) To maintain data integrity by sequencing fragmented transmissions.

As noted above, the design of patient worn transceiver enables the device with hardware components and software modules to provide connectivity over the internet in some embodiments. Remote monitoring, such as via the internet, provides an additional extension to the system to provide additional independent security for the patient.

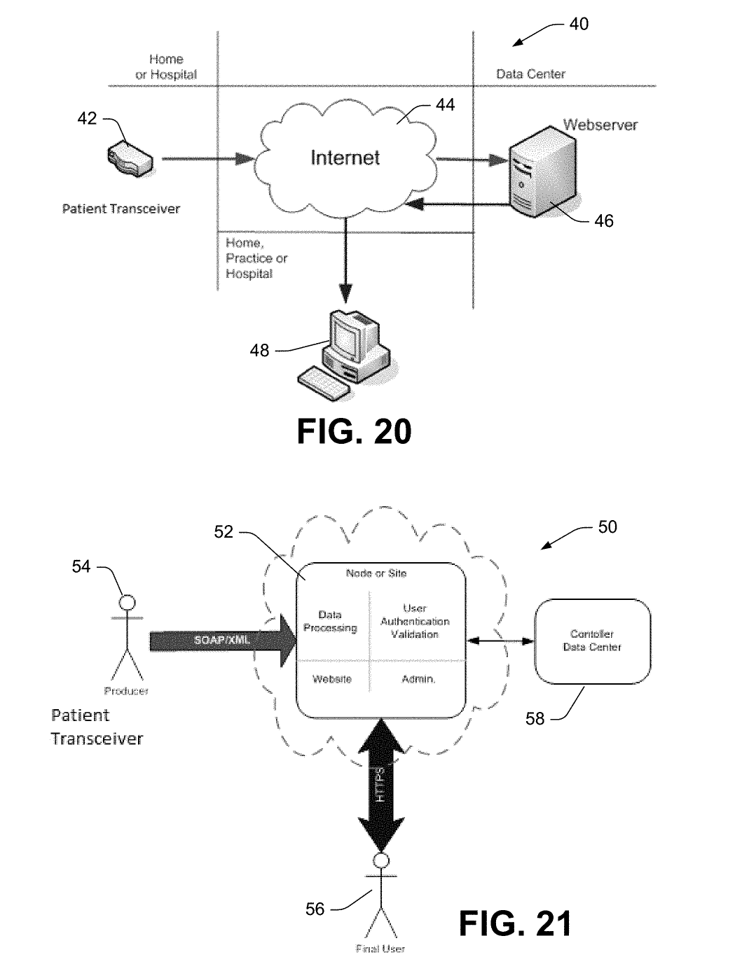

FIG. 20 illustrates an example of an example topology for a remote monitoring system 40. The system 40 includes a patient worn transceiver 42 that communicates with a vascular sensor and sends data over the Internet 44 (either from home or hospital, for example). A webserver 46, located in a datacenter, receives the data from the device. The webserver 46 sends processed data over the internet 44 to a computer client 48 connected to the internet 44. Through the data transmission process, the patient is identified by an alpha-numeric key, no personal information is included in data packets and only the treating hospital or remote monitoring station is able to decode the patient key into actual personal data. The illustrated system 40 includes several decoupled components, usually agnostic to the purpose and without relation between themselves, chained together in a dynamic, time-driven process. The interface process is constrained by narrow timeframe margins imposed by external factors such as network resources, bandwidth, processing speed, latency, etc.

The components involved in a remote monitoring system are identified in categories:

| Component | Description |
|---|---|
| Generators | Devices producing data in real-time (such as the patient transceiver) |
| | and devices that generate post-processing analytic data |
| Networking | Includes all the infrastructure, resources, service providers, |
| | intermediate forwarders and communication protocols involved in |
| | the transmission of computer data between two points. |
| Nodes (or Sites) | Represents the minimum logical unit on the network, provides |
| | functionality to authenticate, validate and process information. |
| | Interacts with devices, users and the system core node. |
| Distributors (or | Includes methods and protocols to transmit data (raw, formatted or |
| Channels) | post-processed information) from a Data Center to a destination. |
| | For example: |
| | To a Website (Internet) |
| | To an Electronic Medical Record (EMR) service |
| | To a database engine |
| | To computer systems (data bridging) |
| | To an external backup service (warehousing) |
| Users | Defines the human component, final recipients. Individuals such as |
| | Physicians, Technicians, Care givers, etc. and terminal equipment |
| | (such as desktops, laptops, PDA, mobiles, etc. where the |
| | information is requested from a channel and visualized on-demand) |

Depending on the configuration level, the remote monitoring system provides basic and advanced features; the following table details feature for the preferred embodiment:

| Group | Description |
|---|---|
| Basic | Encrypted data capture and processing |
| Basic | Secured login/password authentication |
| Basic | Web distributor (website). |
| Basic | SSL Connectivity |
| Basic | Site Administrator Interface |
| Basic | Localization (multi language) |
| Basic | User data download |
| Basic | Data storage |
| Basic | Parametric alarm notifications |
| Advanced | Post-processing filtering and data analysis |
| Advanced | Distributor linkage |
| Advanced | EMR distribution |
| Advanced | Physician Exchange (interactive forums) |

The system in the illustrated embodiment is designed as a compartmentalized, Multi-Node network; each node (or “Site”) includes a website as sole distributor, identified by a unique sub-domain code under the domain.subdomain.com secondary internet domain hierarchy.

FIG. 21 depicts a minimum operational unit 50 on the network. Each site 52 (usually associated with a particular geographic location) concentrates producers 54 and users 56 for a particular attraction area into an individual entity. Each site 50 implements functionality for:

| Functionality | Description |
|---|---|
| Data | Handles connections from patient transceiver devices |
| processing* | specifically configured to connect to the site. |
| | Performs decoding/decrypting |
| | Converts data into suitable output formats |
| Security and | Validates connections from users explicitly authorized to |
| Administration | access the site thru an external protected authentication |
| | service. |
| | Also provides a Rich Interface application for site |
| | administrators to perform simple administrative tasks, |
| | such as: |
| | user management, |
| | device-to-user linkage, |
| | device inventory management |
| Data | Provides a website with protected Rich Interface |
| Visualization | Applications (RIA) to replicate the patient transceiver |
| | screen in quasi real-time. |

In relation to the patient transceiver 42, the site 50 acts as a passive subject, the device initiates communications and the system acknowledges transmissions.

Each site 52 is configured to validate connections from a list of pre-approved patient transceiver devices; the patient transceiver transmits encoded packets at certain frequency, to the site 52 where it is decoded and processed based on configuration parameters. The client/server nature of this communications uses industry standard protocols (SSL/SOAP/WL) to carry data payloads following a stage process:

Registration: a patient transceiver then registers itself to the website to initiate a sequential connection process, the site validates the request (authenticates the connection using an external site authentication service) returning a grant/deny response.

Start: Once validated, the patient transceiver emits a start message to signal the beginning of transmissions, the system returns a go/no-go reply message.

Data: once allowed to begin transmissions, the patient transceiver enters transmission mode, sending data packets to the site, each packet is decrypted and temporarily stored for further delivery to a channel. The system configuration defines an expiration parameter to purge packets from temporary storage (whether the packet was relayed or not to a distributor)

The site contains functionality to validate connection requests from both devices and users; the actual authentication occurs at a protected central location (the controller data center 48 shown in FIG. 21), the security feature of the site acts upon the result of the authentication.

Patient Transceiver Validation: based on database and configuration parameters, allowing or denying the device to continue transmissions to the site.

User Validation: based in login/password pairs, also extends to database parameters to grant/deny the ability to display active device information. A user is restricted, not only to the site, but also to a subset of authorized devices linked to the user by the Site administrator.

The security module also provides a rich interface application (MA), available to selected users (“Site Administrators”), to execute simple administrative tasks:

Add/Remove users

Enable/Disable access to users.

Grant/Deny access to devices by users.

The RIA technology delivers high-quality dynamic graphical user interfaces, commonly referred as dashboards. RIA architecture also provides an additional security layer, as embedded objects in an html page, a RIA runs as a separate, domain-restricted application by creating its own set of secured channels to interact with the site.

A colorful replication of the patient transceiver screen gives the user a realistic display while reproducing the data as transmitted by the device, also adding web-specific features for the user.

A datacenter hosts one or more, geographically related sites. The patient transceiver devices connect to a site to deliver data (fallback to a datacenter). Users connect to the site to visualize data, and each site connects to a designated datacenter (fallback to a controller datacenter). The datacenter provides authentication/validation, monitoring and other features to the site. Each datacenter links to a controller datacenter to provide redundancy and fallback features.

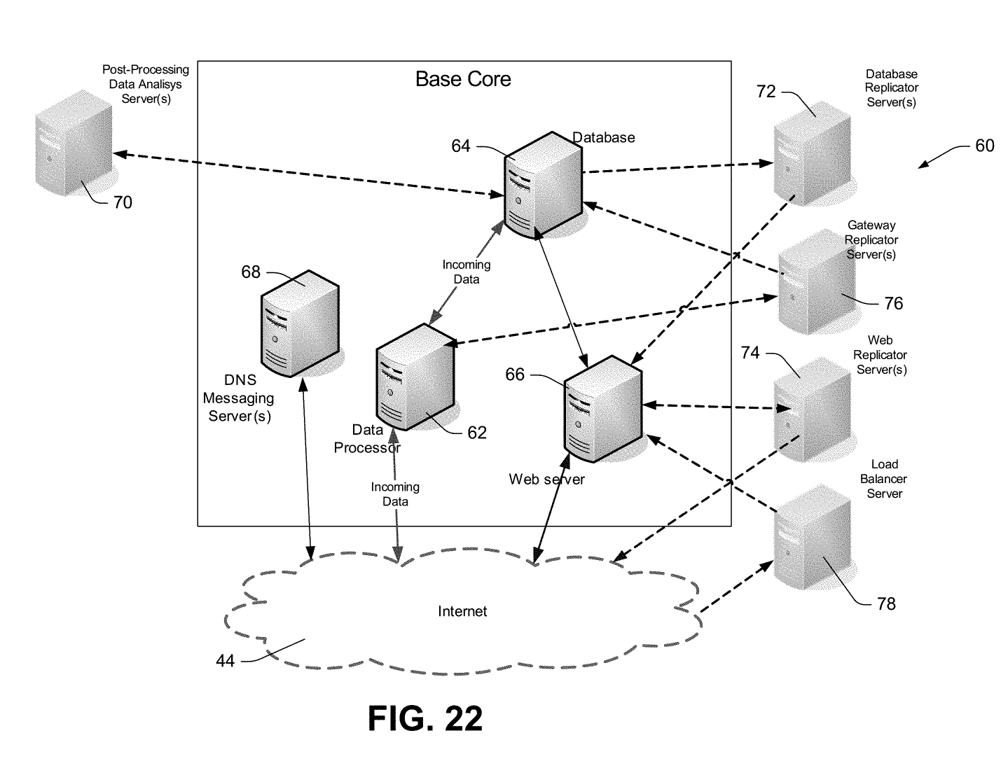

FIG. 22 illustrates an exemplary data center topology 60. In order to implement functionality as describe in earlier sections, each datacenter is initially modeled using a set of base core components including:

| Component | Description |
|---|---|
| Data Processor | Handles patient transceiver connections and |
| Server 62 | processes incoming data. |
| Database server 64 | Stores transient data for further delivery to a channel |
| Web Server 66 | Handles user connections and on-demand data |
| | requests. |
| DNS and Messaging | Auxiliary server to maintain a pool of Domain |
| server 68 | Name servers answering requests from the internet. |
| | Also provides real-time messaging and |
| | communications services (email, SMS, Phone alerts) |

Depending on service level, operating conditions, transaction volumes, interne health and other factors, the base core topology is expanded to include optional components, one or more of the same kind, to increase processing power, including:

| Optional Component | Description |
|---|---|
| Post-processing servers | Provides post-process data analysis |
| 70 | |
| Database servers 72 | Replicates master database servers to provide |
| | redundancy and data extraction load-balancing |
| Web Server 74 | Increases concurrent user service capability |
| Data Processors servers | Increases data processing capabilities when |
| 76 | transmitting devices produce high-volume |
| | processing peaks. |
| Load Balancer server 78 | Provides a programmatic interface to redirect |
| | request based on usage, increases performance |
| | and resource optimization. |

The remote monitoring system's infrastructure in disclosed embodiments is protected by several security mechanisms including firewalls, encryption, login/password routines, SSL protocol implementations and other features. Although, invisible to the user, and not available for outsiders, embedded monitoring features allow the network administrators to detect intrusion attempts, trace usage and system status.

The patient transceiver remote monitoring system situates its network components in high-performance facilities, equipped with self-sustained power-failure recovery, regulated room temperature, structured cabling and restricted access.

Thus, various examples disclosed herein provide an implantable sensor for detection of patient conditions such as vascular wall disease, emboli, Intimal Hyperplasia (stenosis), atherosclerotic plaque, blood vessel thickening, blood vessel thinning. The disclosed sensor may include a catheter based fiber optic coupled sensor for emboli detection. In some examples, sensor operations include introducing source waves into the blood vessel and detecting characteristics of returning waves to differentiate between baseline (patent) vessels and vessels with conditions such as thrombus (said waves consisting of EM waves, sound, etc.). Patient conditions may be detected based on detecting changes in a native blood flow pulse waveform due to presence of a clot in the blood vessel.

Further, source waves (EM waves, sound, etc.) may be introduced into the blood vessel, wherein characteristics of returning waves may be detected to differentiate between a baseline blood vessel wall and altered vessel wall. Conditions such as emboli, and changes in native blood flow pulse waveform, for example, due to altered blood vessel wall may be detected in this manner.

The sensor may be utilized in an Extravascular manner for Short-term (peri-procedural emboli) and Long-term (other emboli, DVT, intimal thickening & plaque) indications. The sensor may be utilized in an Intravascular manner (increased thrombosis risk) Short-term (peri-procedural emboli) and Long-term (other emboli, DVT, intimal thickening & plaque): Smart Stents indications. The sensor may be configured for Continuous sensing for Emboli detection or the sensor may be configured for Intermittent sensing DVT, Intimal thickening, plaque, and aneurysm indications.

In some embodiments, processing of sensoric data is performed on a patient worn device, while in other embodiments sensor data is transmitted over the Internet for processing and evaluation by a clinician or clinical bureau.

The sensor may be configured to run continually or periodically. Various power sources may be employed. Vessel pulsatility may be used to generate power to run the sensor through motion harvesting. The sensor may be passively powered by external instrument, and/or internally powered by primary or secondary battery.

A single sensor or multi-sensor array may be configured as a small implantable network. For light-based sensors, PWM control of the light emitter may be employed to lower overall sensor power and to maximize implant life. An automatic light emitter bias control may be used to ensure the receptor always receives an adequate reflected signal amplitude to ensure good signal-to-noise ratio (SNR), and the minimal amount of light output is needed for signal reception in order to maximize battery life and not unnecessarily overdrive the light emitter. Instantaneous signals may be used to detect some conditions—i.e. blood clot going to a major organ: brain, lungs, kidney, heart, etc., while longer term (days or weeks) of signal changing due to a build-up of a clot may be analyzed to detect other conditions such as deep vein thrombosis, vascular stenosis, atherosclerosis, aneurysm. Visible spectrum light or non-visible spectrum light may be used.

In embodiments using an implanted device, the device may be situated around an artery or vein, including the pulmonary artery, aortic arch, femoral vein, carotid artery. In other embodiments, an external device is used relative to the carotid artery, for example.

The sensor could provide a raw signal out to external equipment and/or have on-board signal processing. The patient or clinicians may be alerted with external hardware, or through a smart phone app for example. In some implementations, sensor data is first transmitted through the body to a patient worn transceiver which may be programmed to process said data with expert algorithms and provide indication to the patient through a user interface as to the presence of a clot such that he/she can immediately seek clinical care. Alternatively, sensor data may first be transmitted through the body to a patient worn transceiver and subsequently may be transmitted further through a wired or wireless intranet or Internet connection to a remote station for processing, recording, evaluation, and disposition by a set of expert algorithms, trained clinical expert, or a combination thereof.

Various signal analyses have been disclosed, including time domain analysis (amplitude threshold, peak-to-average (P/AR), peak-to-rms (Crest Factor)) detection algorithms for detecting occurrence and number of blood clots, as well as frequency domain analysis and phasor domain analysis.

Various modifications and alterations of this disclosure may become apparent to those skilled in the art without departing from the scope and spirit of this disclosure, and it should be understood that the scope of this disclosure is not to be unduly limited to the illustrative examples set forth herein.

What is claimed is:

1. A vascular sensor system, comprising:
a vascular sensor configured to monitor a blood vessel parameter and to output a signal representative of the measured parameter;
a computing system configured to receive the signal representative of the measured parameter, wherein the computing system is programmed to process the received signal and evaluate a medical condition based on the processed signal.

2. The vascular sensor system of claim 1, wherein computing system is configured to communicate with a remote monitoring device.

3. The vascular sensor system of claim 1, wherein computing system includes a patient-worn device.

4. The vascular sensor system of claim 1, wherein the sensor is implantable.

5. The vascular sensor system of claim 1, wherein the sensor includes a light source configured to emit light for illuminating a monitored blood vessel, and a photodetector configured to receive the emitted light that is scattered and/or reflected by the monitored blood vessel.

6. The vascular sensor system claim 5, wherein the light source is configured to emit near infrared (NIR) light.

7. The vascular sensor system claim 5, wherein the light source includes a light emitting diode (LED).

8. The vascular sensor system claim 5, wherein the light source includes a laser.

9. The vascular sensor system of claim 1, wherein the computing system is programmed to process the received signal to differentiate between baseline vessels and vessels with thrombus.

10. The vascular sensor system of claim 1, wherein the computing system is programmed to process the received signal to detect changes in a native blood flow pulse waveform due to an altered blood vessel wall of a monitored blood vessel.

11. The vascular sensor system of claim 1, wherein the computing system is programmed to process the received signal to detect emboli.

12. A vascular monitoring method, comprising:
situating a vascular sensor proximate a monitored blood vessel, the vascular sensor configured to monitor a blood vessel parameter and to output a signal representative of the measured parameter;
processing the signal representative of the measured parameter to evaluate a medical condition based on the processed signal.

13. The method of claim 12, further comprising outputting the signal representative of the measured parameter to a computer system wherein the computing system is programmed to processing the signal representative of the measured parameter to evaluate the medical condition based on the processed signal.

14. The method of claim 12, further comprising implanting the vascular sensor in a patient.

15. The method of claim 12, wherein the sensor includes a light source and a photodetector, and wherein the method further comprises:
illuminating the monitored blood vessel with the light source;
receiving the emitted light that is scattered and/or reflected by the monitored blood vessel by the photodetector; and
wherein the signal representative of the measured parameter is based on the received light.

16. The method of claim 12, wherein processing the signal includes differentiating between baseline vessels and vessels with thrombus.

17. The method of claim 12, wherein processing the signal includes detecting changes in a native blood flow pulse waveform due to an altered blood vessel wall of a monitored blood vessel.

18. The method of claim 12, wherein processing the signal includes a time domain analysis.

19. The method of claim 12, wherein processing the signal includes a frequency domain analysis.

20. The method of claim 12, wherein processing the signal includes a phasor domain analysis.
